(19) United States
(12) Patent Application Publication
(10) Pub. No.: US 2026/0062743 A1
(43) Pub. Date: Mar. 5, 2026

(54) DETECTION OF MINIMAL RESIDUAL DISEASE FROM SURGICAL DRAIN FLUID

(71) Applicant: Droplet Biosciences, Inc., Cambridge, MA (US)

(72) Inventors: Zhuosheng Gu, Jersey City, NJ (US); Wendy Winckler Adamovich, Melrose, MA (US)

(21) Appl. No.: 19/313,338

(22) Filed: Aug. 28, 2025

Related U.S. Application Data
(60) Provisional application No. 63/688,050, filed on Aug. 28, 2024.

(51) Int. Cl. C12Q 1/6869; C12Q 1/6886

(52) U.S. Cl. CPC C12Q 1/6869; C12Q 1/6886; C12Q 2600/106; C12Q 2600/118

(57) ABSTRACT

The present invention provides methods for detecting and predicting minimal residual disease in a subject who has undergone tumor resection surgery. Methods of the invention utilize a personalized cutoff for determining the likelihood for MRD.

### TECHNICAL FIELD

The invention provides methods for determining minimal residual disease from surgical drain fluid.

### BACKGROUND

Cancer results from complex and heterogenous molecular derangement. Early detection, while beneficial for most cancers, is often difficult. Further, for patients undergoing surgical treatments of various cancers, there are currently no completely effective methods to determine if cancer cells were left behind. As a result, physicians must define post-surgery treatments such as radiation or chemotherapy, based on limited information. This endangers patient outcomes and quality of life.

When patients are diagnosed with localized cancer, the standard of care typically starts with surgical resection of the tumor. For many patients, resection is successful. However certain aggressive tumors, like head and neck squamous cell carcinoma (HNSCC), the sixth most common cancer, have an increased risk of tumor recurrence.

Minimal residual disease (MRD) refers to the small number of cancer cells that remain in the body after treatment. Detecting minimal residual disease may indicate that a treatment was not completely effective or that the treatment was incomplete, potentially because not all of the cancer cells responded to therapy.

One focus of cancer detection is liquid biopsy in blood or plasma. Blood is of high clinical interest because of its accessibility. Yet, the detection of plasma analytes is ill-suited for post-surgery monitoring of residual disease.

First, given the systemic nature of circulation, the detection of cancer biomarkers in plasma is non-specific and does not provide the ability to determine whether the source of the cancer biomarker (i.e., the cancer) is the surgical site. Second, the dilution of analytes within a patient's plasma volume results in low concentrations of analytes in plasma samples, thereby increasing the challenge of detection. Finally, the relatively long latency of the appearance of biomarkers in the plasma results in a temporal lag between the release of biomarkers from the surgical site and their appearance in plasma. Accordingly, identifying patients who would most benefit from escalated treatment from those who may be de-escalated remains a critical need.

Liquid biopsy, particularly cell-free DNA based diagnostics, have shown clinical utility in guiding treatment for non-squamous cell lung cancer (NSCLC), breast cancer and colorectal cancer. In HNSCC, post-surgical plasma ctDNA detection has been associated with clinical recurrence. Utilizing biofluids proximal to the tumor has shown increased sensitivity compared to plasma across many cancer types. As a consequence, there is a need for sensitive and specific molecular diagnostics for residual disease.

### SUMMARY

The invention provides methods for evaluating minimal residual disease. Some embodiments involve sequencing nucleic acid from a biological fluid to determine a number of mutations at a defined depth of coverage to establish a cutoff value for establishing or calling minimal residual disease (MRD). The cutoff is determined on a patient-specific basis and may be used in subsequent assays to analyze samples, evaluate MRD, select therapeutics, and/or evaluate therapeutic efficacy.

According to one embodiment, the invention provides a personalized cutoff for MRD that takes into account somatic mutation count and an average per base depth of mutations detected in a sample containing ctDNA. The cutoff provides a patient- and sample-specific metric for minimal residual disease. Accordingly, methods of the disclosure take into account both tumor mutational burden (TMB) and mutation detection coverage for each patient when determining an MRD cutoff. Accordingly, the disclosed methods increase the accuracy of detection and minimize any outlier bias that results from either a specific patient's high TMB or high mutation coverage.

For example, in conventional methods, high TMB results in a greater number of non-detected mutations in the sample as compared to low TMB. This results in an artificially lower variant allele frequency (VAF). Similarly, higher mutation coverage will also result in a lower variant allele frequency. In either case, the likelihood of a false negative result is increased. Embodiments of this disclosure address these problems by providing a customized or personalized cutoff for each patient and/or sample.

In one embodiment, methods of the invention take into account TMB and mutation coverage for a specific patient in determining an MRD cutoff for additional samples from the patient. In one embodiment, the MRD cutoff is defined as a product of the inverse of somatic mutation count and mutation sequencing depth in a patient sample. The result is a quantitative cutoff to determine the presence of minimal residual disease in a specific patient and/or sample. Accordingly, in one or more embodiment, the metric for MRD cutoff (MC) is defined as

$$M C = \frac{2}{{N}_{t m}} \times \frac{2}{D {P}_{m}}$$

where Nₜₘ is somatic mutation count in a patient sample and DPₘ is depth of coverage of Nₜₘ mutations in the sample. The term,

$$\frac{2}{D {P}_{m}}$$

is roughly equivalent to the limit of detection or minimum variant allele frequency that can be detected in the sample.

In alternative embodiments, the cutoff (MC) can be determined using a numerator other than 2. The inventors have found that the choice of numerator will affect the sensitivity and specificity of the metric. For example, if the numerator is less than 2, sensitivity will go up at the expense of specificity. Using a numerator higher than 2 will have the opposite effect.

In essence, the invention provides methods for balancing depth of coverage and TMB to inform an MRD cutoff. More importantly, balancing mutation load and depth of coverage in a patient-derived sample results in a patient- and sample-specific determination of the likelihood of MRD. In one instance, the cutoff provides a diagnostic cutoff for MRD.

Methods of the invention are applicable to any tissue or body fluid (e.g., blood, urine, saliva, sputum, pus, and the like). In one embodiment, a sample is obtained from surgical drain fluid collected during or after a surgical procedure.

In one example, drain fluid may be collected at the time of intervention and then periodically over the course of hours, days, or weeks. The surgical procedure may be a procedure that is not related to a disease being diagnosed.

Some embodiments of this disclosure provide sensitive and specific diagnostics that allow assessment of disease such as staging, status, and/or progression. Some embodiments of this disclosure aid in therapeutic selection and/or assessment of therapeutic efficacy.

Lymph fluid plays a critical role in the tumor microenvironment and is the primary route of metastatic spread. However, this biofluid is largely inaccessible due to difficulty in visualizing and accessing lymph vessels. Following tumor resection, lymphatic fluid (lymph) can be accessed via routinely placed surgical drains. Since drain fluid may comprise a combination of irrigation fluid, blood, plasma cells, and lymphatic fluid, it may be necessary to isolate a fraction of interest from the drain fluid. Embodiments of this disclosure contemplate the use of both raw drain fluid and isolated fractions thereof. It is noted however that raw drain fluid contains a sufficient amount of diagnostic content to be used in embodiments of this disclosure without further sample preparation.

Some embodiments of the disclosure are also applicable to therapeutic selection and/or therapeutic efficacy. In one or more embodiment, a number of mutations is determined, the calculated cutoff value for minimal residual disease is determined, and a therapeutic agent is selected and/or evaluated for efficacy based on the calculated cutoff. In some embodiments, a therapy is administered and the quantity of mutations compared to the cutoff value after administration. In some embodiments, these methods are performed using the nucleic acids from drain fluid.

### BRIEF DESCRIPTION OF THE DRAWINGS

FIGS. 1A-1B show Kaplan-Meier survival curves demonstrating results of one or more embodiment of this disclosure.

FIGS. 2A-2C show data from various downsampling seeding techniques according to one or more embodiment of this disclosure.

FIG. 3 shows the results of the disclosed dynamic MRD cutoff calculations across a cohort of 73 patients.

### DETAILED DESCRIPTION

The present invention provides methods of liquid biopsy-based diagnostics for cancer recurrence and/or MRD. In some embodiments, the utilization of non-systemic bodily fluids provides the ability to measure locoregional minimal residual disease as well as determine a risk of recurrence.

In one aspect, the present invention provides methods to determine a patient-specific cutoff for minimal residual disease. Some methods include determining a number and depth of mutations in nucleic acid from a patient. The number and depth of mutations in the sample are used to establish a customized cutoff for evaluation of MRD.

Methods of the invention are applicable to any liquid biopsy sample and any form of cancer. In a some embodiments, methods of the invention are applied to a surgical drain fluid sample. Surgical drain fluid may be obtained from any medical intervention, including surgeries, biopsies, catheterizations, dissections, intubations, and the like. Often, as part of a postoperative regime, patients receive an implanted surgical drain, such as a Jackson-Pratt (JP) drain, which removes fluid (including lymph fluid) that collects at the site of a surgery. These surgical drains that are routinely placed after tumor resection to prevent fluid build-up and speed healing are a previously unrecognized source of lymphatic exudate. Such drains are employed in majority of primary cancer surgeries and the exudate obtained from such drains is proximal to the tumor bed as well as adjacent lymph nodes. Adjacent lymph nodes to the tumor are usually the locations of micro-metastases and are likely to have an increased concentration of tumor-associated analytes in the setting of minimal residual disease. Therefore, proximal bio-fluids are extremely enriched for tumor associated mutations and provide more sensitive detection of locoregional minimal residual disease.

Surgical drain fluid is rich in diagnostic information due to presence of certain biomarkers indicative of disease. The biomarker may be a nucleic acid (DNA and/or RNA), a protein, any other molecule or compound, or a cell. Biomarkers useful in the invention vary and may be selected based on the disease indication being treated/monitored and other factors known to the skilled artisan. Moreover, sensitivity and specificity may vary across biomarkers and that will influence biomarker selection.

Examples of suitable biomarkers or fluid components that may be collected using methods of the invention include tumor cells, immune cells, bacterial cells, viral host cells, donor organ cells, microvascular cells, cell-free DNA (cfDNA), cell-free RNA (cfRNA), circulating tumor DNA (ctDNA), messenger RNA, exosomes, proteins, hormones, and analytes. In certain embodiments, the biomarker may be one or more of interleukin-1, interleukin-6, interleukin-10, a tumor necrosis factor, matrix metalloproteinase-1, matrix metalloproteinase-2, matrix metalloproteinase-9, matrix metalloproteinase-13, or a nucleic acid comprising a mutation. In one embodiment, the biomarker of choice is a nucleic acid, particularly cell-free DNA.

In various aspects, biomarkers may be identified and quantified using methods known in the art. Suitable assays may include, for example, nucleic acid sequencing, PCR, quantitative PCR, digital droplet PCR, Western blot target capture, proteomics, nucleic acid expression analysis, and antibody screening.

Some methods of the present invention determine mutations in cell-free DNA by sequencing the DNA. The number of mutations and depth of coverage are determined, processed, and compared to a predetermined cutoff value for minimal residual disease specific to the individual patient. For example, assays may include whole genome sequencing, next generation DNA sequencing, next generation RNA sequencing, multiplex PCR, methylation analysis, droplet PCR, droplet cell separation, or any combination thereof. In one aspect of the invention, the cell-free DNA is sequenced using next generation sequencing to determine the number of mutations in the cell-free DNA from a drain fluid.

Methods taught herein also provide sensitive and specific diagnostics that aid in therapeutic selection and efficacy. The invention provides methods for initial diagnostics comprising steps of sequencing nucleic acid from surgical drain fluid of a subject to determine a number of mutations in the nucleic acid from the drain fluid; and comparing the determined number of mutations with a pre-calculated cutoff value for minimal residual disease for the subject and providing to the subject a therapeutic agent if the number of mutations above the pre-calculated cutoff value for minimal residual disease. Accordingly, a putative therapeutic may be selected or evaluated based on the cutoff value or the number of mutations present in a sample after administration of the therapeutic.

### Example 1

A study was conducted to demonstrate the effectiveness of methods of the invention. Human subjects with HPV-negative HNSCC were recruited for participation. Clinical data were obtained from electronic health records (EHRs) of the enrolled subjects. Baseline clinical and pathological features were recorded upon patient enrollment in the study. Surgical lymphatic fluid (“lymph”) from surgical drain samples, plasma, and peripheral blood samples were collected at 24 hours post-surgery. Resected tumor samples were also collected. Subsequent follow-up data including adjuvant therapy after surgery, treatment outcomes, and dates of disease progression, death, and surveillance follow-up scans were collected periodically from the EHRs of the enrolled patients.

### Sample Collection, Processing and DNA Extraction

All patients underwent Jackson Pratt (JP) surgical drain insertion during surgery. Lymph was collected from JP drains in 50 mL conical tubes approximately 24 hours after surgery. Peripheral blood was collected in vacuum tubes at the time of lymph collection. Tumor tissue was collected after surgery. Fresh-frozen tumor samples were snap-frozen in liquid nitrogen, embedded in optimal cutting temperature (OCT) compound blocks on dry ice and stored at −80° C.

An aliquot of cfDNA was extracted from 1-4 mL of blood plasma using the QIAmp Circulating Nucleic Acid Kit (Qiagen, DE). Lymph cfDNA was extracted using Droplet's proprietary extraction method. 150 L of whole blood or one blood cell pellet was used for genomic DNA extraction with the Qiagen DNeasy Blood and Tissue kit (Qiagen, DE). Tumor tissue DNA was extracted using the QIAmp DNeasy Blood and Tissue kit (Fresh-Frozen) or QIAmp DNA FFPE Kit (FFPE) (Qiagen, DE). DNA concentrations were quantified using the Qubit HS dsDNA Assay (Thermo Fisher Scientific, Waltham, MA). DNA quality was assessed using the Agilent Tapestation Genomic DNA and cfDNA Analysis kits (Agilent Technologies, Santa Clara, CA).

### Next-Generation Sequencing

Genomic DNA was fragmented to 150 bp with the Covaris ME220. 80 ng of cfDNA or genomic DNA was prepared using the xGen cfDNA and FFPE Library preparation kit (Integrated DNA Technologies, Coralville, IA). 600 ng of prepared library was used for hybridization using a custom hybridization panel and standard hybridization reagent kit (Twist Bioscience, San Francisco, CA). Pre-hybridized and hybridized libraries were assessed using DS1000 DNA Screentape (Agilent Technologies, Santa Clara, CA) and quantified using Qubit HS dsDNA Assay (Thermo Fisher Scientific, Waltham, MA).

Hybridized libraries were sequenced on NovaSeq 6000 (Illumina, San Diego, CA). Whole blood and tumor libraries were sequenced to 500× deduplicated coverage. Plasma and Lymph cfDNA libraries were sequenced to 5000× de-duplicated coverage.

### Tumor-Informed Variant Calling

Raw reads were demultiplexed using BCL Convert (Illumina, San Diego, CA). Consensus reads were called with fgbio (Fulcrum Genomics, Boulder, CO) based on UMIs extracted. The reads were aligned to the hg38 human genome using the Burrows-Wheeler Aligner (BWA) as part of the nf-core/sarek pipeline. Coverage analysis was performed using Picard (Broad Institute, Cambridge, MA) on bam files generated.

Mutect2 (Broad Institute, Cambridge, MA) was used to call variants in the tumor samples with matched normals and a panel of normals. The panel of normals was created with 50 PDWB samples following GATK best practices. From these variants, somatic mutations on coding sequences were identified using Ensembl Variant Effect Predictor annotation. All somatic mutations were then used to force call mutations in matched lymph and plasma samples. Matched PDWB were used to filter out germline mutations or mutations driven by age-associated clonal hematopoiesis. A base-specific error model (BEM) was estimated at each somatic mutation position to determine the background noise. Each model was built using a series of high-quality lymph and plasma samples sequenced to >5000× deduplicated coverage. Weibull or Normal distribution was fit using the variant allele frequency (VAF) at each somatic mutation position across these lymph and plasma samples. For each patient, mutations were retained only of they had VAF greater than the BEM cutoff.

### Data Analysis

Python 3.8.10 was used for all subsequent analyses. Mann-Whitney U test was used for group comparisons between lymph and plasma samples, and comparisons between REC and NED patients. A dynamic MRD cutoff (MC) was estimated for each lymph or plasma sample based on its sequencing depth and somatic mutation count in corresponding tumor sample using the formula below:

$$M C = \frac{2}{{N}_{tm}} \times \frac{2}{D {P}_{m}}$$

where Nₜₘ is somatic tumor mutation count of the patient and DPₘ is average per base depth of Nₜₘ mutations in the patient lymph or plasma sample. cfDNA samples with mutations mean VAF greater than the dynamic cutoff were predicted as MRD positive, and those with mean VAF lower than the dynamic cutoff were predicted as MRD negative.

Following binary prediction by the model, the Kaplan-Meier estimator with log-rank test and Cox proportional-hazards model were used for survival analysis. Sensitivity and specificity were calculated based on disease progression status respectively. Student's t-test, Fisher's exact test, and Spearman's rank correlation coefficient were used to rule out confounding effects by clinical and pathological features.

### Study Population

A total of 82 HPV-negative HNSCC patients were included in the study. Ten patients were censored due to lack of clinical data, for a total study population of 72 patients. Eleven patients had no matched plasma samples. Baseline clinical values are shown in Table 1 below.

TABLE 1
Study Population Baseline Clinical and Pathological Characteristics

| | | | | Other high | | | |
|---|---|---|---|---|---|---|---|
| | | | | risk path. | | | |
| Subject | | | | features | Neoadjuvant | Adjuvant | |
| ID | Stage | TNM | ENE | (PNI/LVI) | Tx | Tx | Recurr. |
| DF142 | I | T1N0 | Neg | yes | No | none | No |
| DF165 | II | T2N0 | Neg | no | No | none | Local |
| DF097 | II | T2N0 | Neg | yes | No | none | No |
| DF076 | II | T2N0 | Neg | no | No | none | No |
| DF080 | IVa | T2N2b | Neg | yes | Yes- Pembro | RT | No |
| DF115 | IVa | T2N2c | Neg | yes | No | RT | Local |
| DF157 | III | T3N0 | Neg | yes | No | RT | No |
| DF086 | III | T3N0 | Neg | yes | No | RT | No |
| DF128 | III | T3N1 | Neg | yes | No | RT | No |
| DF081 | IVa | T3N2b | Neg | Ino | No | RT | No |
| DF114 | IVa | T4aN0 | Neg | no | No | RT | No |
| DF064 | IVa | T4aN0 | Neg | yes | No | RT | Local |
| DF179 | IVa | T4aN0 | Neg | yes | No | RT | No |
| DF220 | IVa | T4aN0 | Neg | no | No | none | Local |
| DF072 | IVa | T4aN1 | Neg | no | No | RT | No |
| DF071 | IVa | T4aN2c | Neg | yes | No | none | Local/ |
| | | | | | | | Distant |
| DF068 | IVa | T4aN2c | Neg | yes | No | RT | Local |
| DF181 | IVb | T4aN3b | Pos | yes | No | RT | Local/ |
| | | | | | | | Distant |
| DF208 | IVb | T4aN3b | Pos | yes | No | none | Local/ |
| | | | | | | | Distant |
| DF185 | IVa | T4N2b | Neg | yes | No | none | Local/ |
| | | | | | | | Distant |

ENE, extranodal extension;
LVI, lymphovascular invasion;
PNI, peri-neural spread;
Tx, treatment

After about one year of follow-up, 30 out of 74 patients experienced disease recurrence (REC) and 44 out of 74 patients had no evidence of disease (NED).

Surgical Lymphatic Fluid has a Higher Tumor Allelic Fraction than Plasma

The ctDNA allelic fraction was compared between matched lymph and plasma samples from the study cohort and was found to be 1.5 times higher in lymph than in plasma samples (lymph=0.11±0.16%; plasma=0.076±0.12%; p=0.018, N=100 mutations). Notably, ctDNA was undetectable in only 18.6% of lymph samples compared to 50.9% of plasma samples.

Surgical Lymphatic Fluid ctDNA Positivity Predicts HNSCC Recurrence

The number of mutations both in lymph and plasma samples from patients with REC versus those with NED were compared. It was found that significantly more mutations were detected in patients with REC compared to those with NED (p=0.009). This was not the case for plasma samples, however (plasma REC vs. plasma NED p=0.155).

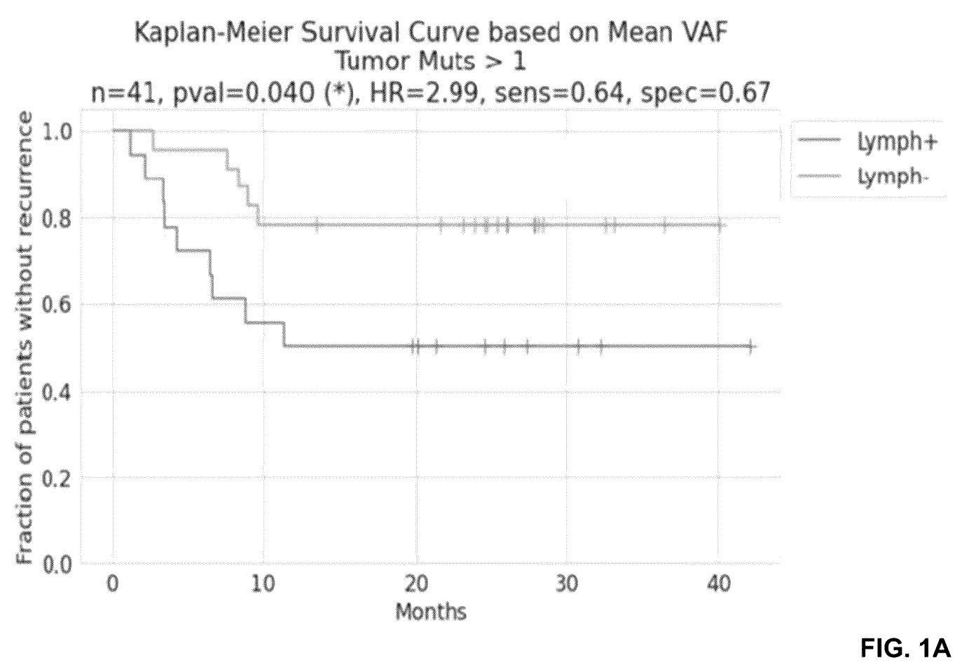

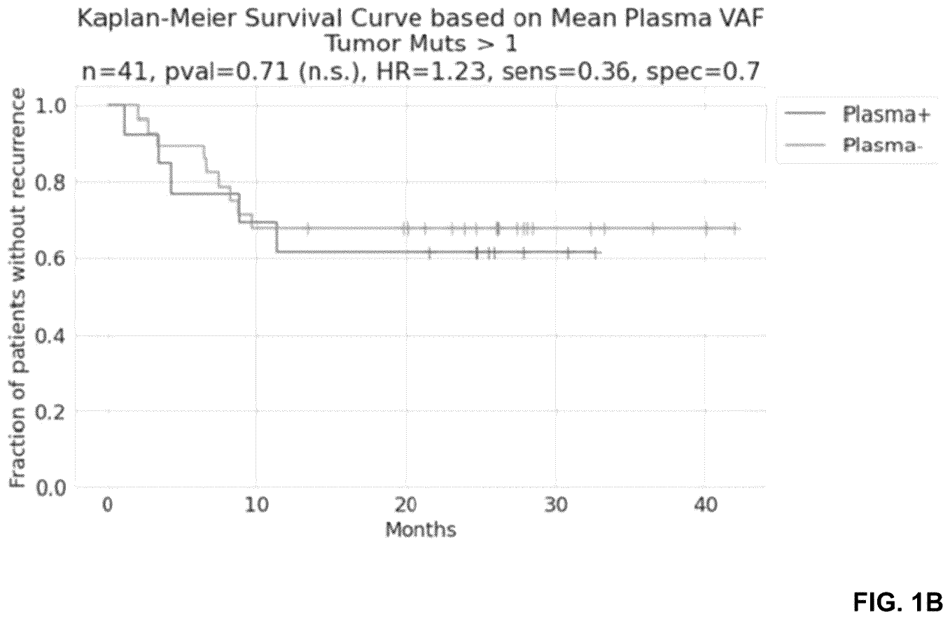

A Kaplan-Meier survival analysis was performed using ctDNA positivity status in both lymph (FIG. 1A) and plasma (FIG. 1B) samples from the study cohort. A sample was classified as positive if two or more force-called tumor-informed mutations were detected. It was found that lymph ctDNA positivity could accurately predict HNSCC recurrence (sensitivity=64%, specificity=67%; p<0.04). whereas plasma ctDNA positivity could not (sensitivity=36%, specificity=70%; p=0.71). The hazard ratio for disease recurrence in patients with ctDNA positivity in lymph samples was 2.99, indicating that disease recurrence was more than 12 times more likely among patients with ctDNA-positive lymph samples than among those with ctDNA-negative lymph samples during the follow-up period.

### Surgical Lymphatic Fluid is Superior to Plasma for the Prognostication of Locoregional Recurrence

Patients were stratified with recurrent disease (n=9) into those with locoregional relapse only (n=4) and those with locoregional plus distant relapse (n=5) and compared the number of mutations detectable in lymph and plasma samples from each group. Among patients with locoregional relapse only, significantly more mutations were detected in lymph samples than in plasma samples (p=0.01). In contrast, similar mutation levels were detected in lymph and plasma samples among patients with locoregional plus distant relapse (p=1.0).

Surgical Lymphatic Fluid ctDNA Accurately Predicts Recurrence in ENE-Negative Disease

The predictive power of ctDNA positivity in surgical lymphatic fluid was compared with that baseline ENE positivity, a high-risk pathologic feature. ENE positivity was concordant with ctDNA positivity in lymph in only 12 out of 20 patients. Thus, 67% of patients with recurrent disease were ENE-negative but ctDNA positive in lymph, suggesting that the latter biomarker may capture patients at higher risk of recurrence despite their having lower-risk pathology.

### Dynamic MRD Cutoff Remains Consistent Despite Variation in Tumor Mutation Count and Sequencing Coverage of Lymph

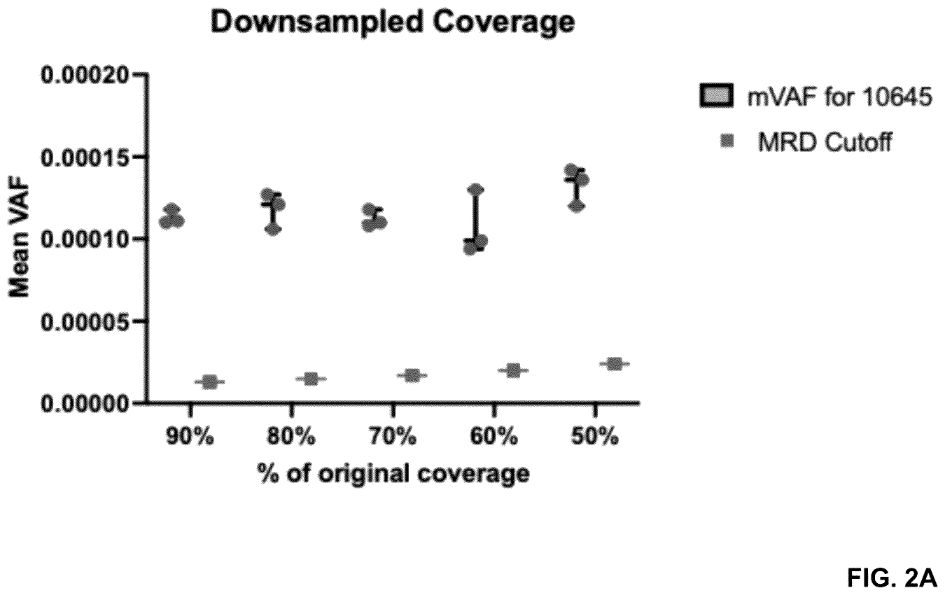

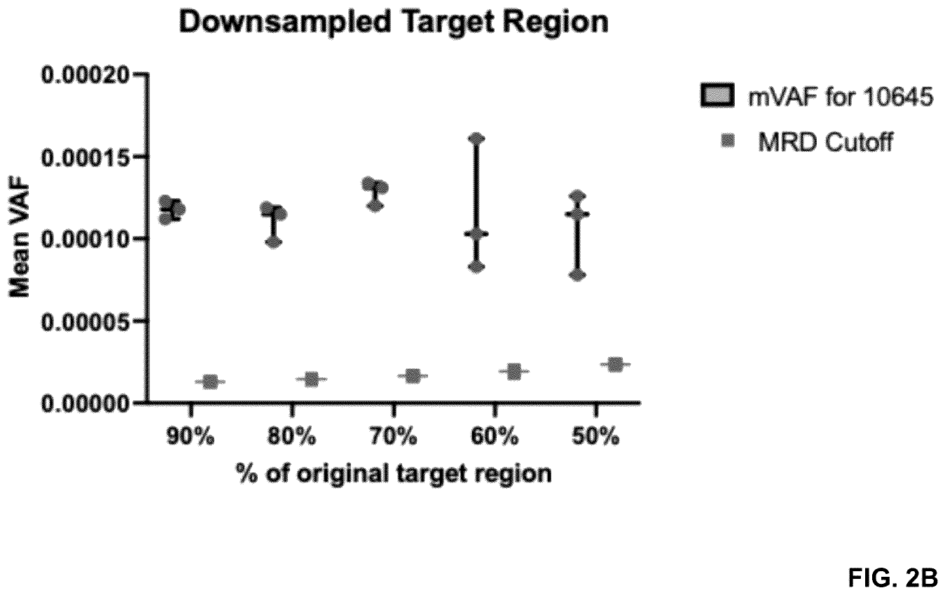

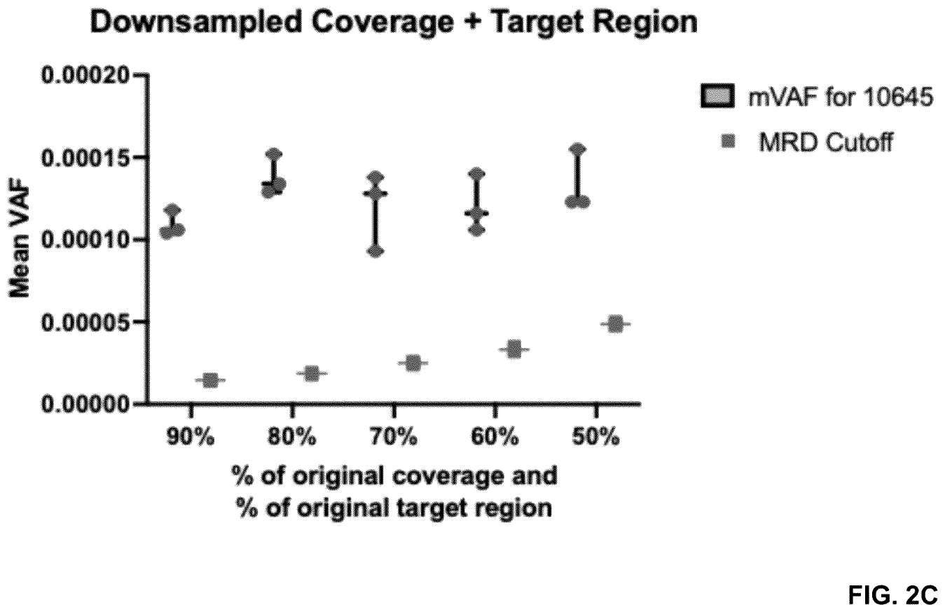

The robustness of the calibrated MRD cutoff was assessed by randomly downsampling one lymph sample with high observed tumor mutation count and coverage. Three technical replicates were created for downsampling to demonstrate consistency with different downsampling seeding. These data, illustrated in FIGS. 2A-2C, indicate that with the use of the coverage- and mutation count-corrected cutoff, consistent and accurate MRD calls were made despite variation in the observed tumor mutation count and achieved sequencing coverage of the lymph sample.

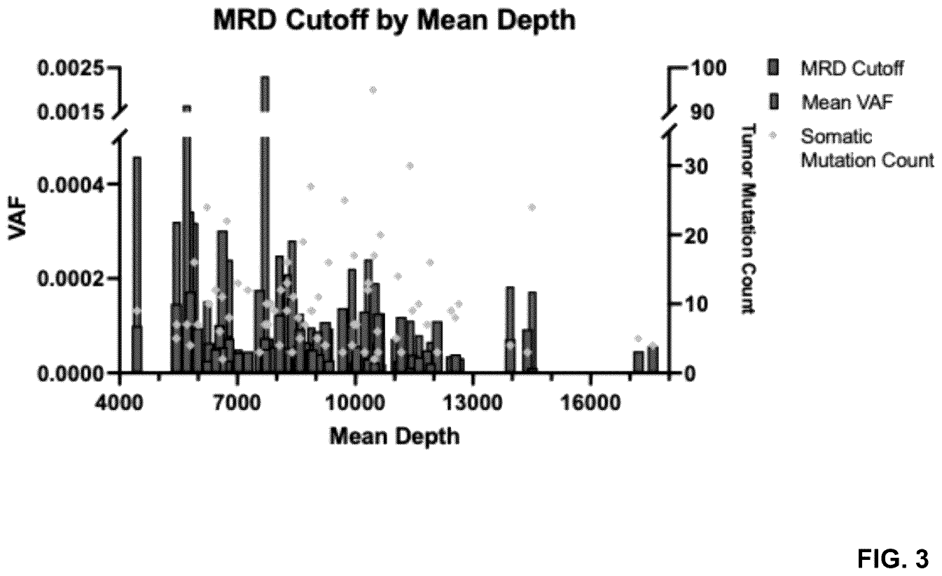

The dynamic MRD cutoff by sequencing coverage across a cohort of 73 patients was plotted in FIG. 3 to demonstrate the combined effect of tumor mutation count and sequencing coverage.

In total, this example demonstrates a novel approach to MRD prediction based upon postoperative ctDNA from surgical drain fluid (lymph) that outperforms conventional liquid biopsy approaches. As show above the ctDNA allelic fraction was about 1.5 times higher in lymph samples versus matched plasma samples; and significantly more mutations were detected in the lymph samples as compared to plasma. Results in lymph demonstrate the ability to accurately predict recurrence with a sensitivity of 89% and a specificity of 82%. Moreover, methods of the invention accurately predict recurrence even in ENE negative samples. Methods described herein are useful in any liquid biopsy sample, but surgical drain fluid provides a superior sample to interrogate locoregional disease.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. A method for establishing a personalized cutoff of minimal residual disease, the method comprising:
determining a depth of coverage of one or more mutations suspected to be present in a liquid biopsy sample obtained from a cancer patient;
sequencing nucleic acid in the sample at the determined depth of coverage to determine a number of a somatic mutation in the sample; and
establishing a personalized cutoff for the cancer patient based upon the number of somatic mutations and depth of coverage,
wherein subsequent results above the personalized cutoff indicate a recurrence of cancer.

2. The method of claim 1, wherein the cutoff is determined as a product of an inverse of the number of somatic mutations and the depth of coverage.

3. The method of claim 1, wherein the liquid biopsy sample is drain fluid from a surgical intervention.

4. The method of claim 3, wherein the surgical drain fluid is obtained via a drain inserted during surgery.

5. The method of claim 3, wherein the surgical drain fluid is obtained by irrigation during a surgical intervention.

6. The method of claim 3, wherein the subsequent sample is collected post-surgery.

7. The method of claim 1, wherein the nucleic acid is ctDNA.

8. The method of claim 1, wherein the cancer is head and neck squamous cell carcinoma.

9. The method of claim 1, further comprising selecting a therapeutic based on the personalized cutoff.

10. The method of claim 9, further comprising administering the selected therapeutic to the patient.

11. The method of claim 1, further comprising:
administering a therapeutic to the cancer patient after establishing a personalized cutoff;
determining a number of mutations present in a subsequent sample after administering the therapeutic; and
comparing the number of mutations to the personalized cutoff to evaluate the efficacy of the therapeutic.
